(19) United States
(12) Patent Application Publication
(10) Pub. No.: US 2004/0223963 A1
(43) Pub. Date: Nov. 11, 2004

(54) Method for treating ischemic stroke with melatonin

(76) Inventors: Raymond Cheung, Hong Kong (CN); Shiu-Fun Pang, Hong Kong (CN)

Correspondence Address: Charles E. Miller, DICKSTEIN SHAPIRO MORIN & OSHINSKY LLP, 41st Floor, 1177 Avenue of the Americas, New York, NY, 10036-2714, US

(21) Appl. No.: 10/770,371

(22) Filed: Feb. 2, 2004

Related U.S. Application Data
(60) Provisional application No. 60/443,918, filed on Jan. 31, 2003.

(51) Int. Cl. A61K 38/46; A61K 31/405

(52) U.S. Cl. 424/94.65; 514/414; 514/565

(57) ABSTRACT

The present invention is directed to a method of treating a sudden onset of at least one neurological deficit in a subject. The sudden onset of neurological symptoms is an indicator of a possible stroke, also termed a cerebrovascular accident. The method comprises administering an effective amount of melatonin to the subject immediately after the sudden onset of at least one neurological deficit, and preferably within three hours of the sudden onset of the at least one neurological deficit. Preferably, the effective amount of melatonin is at least about 200 mg and less than about 1000 mg, although for a small child or infant the effective amount of melatonin may be less than about 200 mg while for a large adult it may be more than about 1000 mg. It is expected that the effective amount of melatonin is no more than about 1500 mg in almost all cases.

### REFERENCE TO RELATED APPLICATION

This application claims the benefit of U.S. Provisional Application No. 60/443,918 filed Jan. 31, 2003.

### BACKGROUND OF THE INVENTION

Stroke is a cardiovascular disease affecting the blood vessels supplying blood to the brain. There are four main types of stroke: two caused by blood clots or other particles, and two by hemorrhage. By far the most common causes for strokes are cerebral thrombosis and cerebral embolism, which are caused by clots or particles that plug an artery. The remaining two are cerebral and subarachnoid hemorrhages caused by ruptured blood vessels.

Stroke, resulting in death of tissue, is the third leading cause of death and a major source of disability in the developed countries and regions. Typically, ischemic damage, i.e., due to lack of oxygen, due to a disruption of the blood supply to a region in the brain is diagnosed as a stroke when accompanied by neurological or other symptoms. In an ischemic stroke focal ischemia exhibiting a defined region of tissue damage is observed, which is often surrounded by a penumbral region that is susceptible to additional damage over time.

The blood supply disruption resulting in a stroke may be due to, inter alia, presence of a blood clot, arteriosclerosis, artherosclerotic plaque (or its components), and the like. Thus, treatment for a stroke has to be, preferably, provided rapidly to avoid irreversible damage. The treatment also has to be in agreement with the underlying cause because, for instance, administering agents to inhibit blood coagulation in a stroke due to a hemorrhage risks increasing the damage by promoting hemorrhage. If the stroke is due to the presence or formation of a blood clot, then treatments are directed to dissolve or otherwise reduce the clots. Some treatments for ischemic stroke include intravenous thrombolysis using tissue plasminogen activator within three (3) hours of onset, acute defibrinogenation using intravenous modified viper venom within three (3) hours of onset, or intra-arterial thrombolysis using prourokinase within six (6) hours of onset.

Acute thrombolysis or defibrinogenation is feasible in less than 5% of stroke patients, and there is a substantial risk of symptomatic hemorrhage into the acute infarct. For example, intravenous thrombolysis carries a ten-fold risk of symptomatic hemorrhage into the acute infarct, and patients with this complication have a mortality rate of 60%. Nevertheless, currently, intravenous thrombolysis is the only acute stroke therapy approved by the Food and Drug Administration of the U.S.A.

Although enhancement of the tolerance of cerebral tissue to ischemia/reperfusion injury has been a goal to complement or replace agents that restore or promote blood flow, clinical trials have so far failed to identify a safe and effective neuroprotectant. Promising neuroprotectant candidates that do not cause unacceptable adverse side effects are almost non-existent as can be seen from the following summary of various clinical trials.

TABLE
Results from clinical trials on neuroprotection against ischemic stroke

| | | |
|---|---|---|
| Drugs | Mode of action | Results |

CALCIUM CHANNEL ANTAGONISTS

| | | |
|---|---|---|
| Nimodipine | Voltage-dependent calcium antagonist | No efficacy |
| Flunarizine | Voltage-dependent calcium antagonist | No efficacy |
| Isradipine | Voltage-dependent calcium antagonist | No efficacy |

NMDA-TYPE GLUTAMATE RECEPTOR ANTAGONISTS

| | | |
|---|---|---|
| Selfotel | Competitive NMDA antagonist | Trial stopped, adverse effects |
| Aptiganel | Non-competitive NMDA antagonist | Trial stopped, adverse effects |
| Dizolcipine | Non-competitive NMDA antagonist | Trial stopped, adverse effects |
| Dextrorfan | Non-competitive NMDA antagonist | Trial stopped, adverse effects |
| Racemide | Non-competitive NMDA antagonist | Plan for phase III |
| Magnesium | Non-competitive NMDA antagonist, | Ongoing phase III |
| | voltage-dependent calcium antagonist | |
| GV150526 | Glycine site antagonist | No efficacy |
| Eliprodil | Polyamine site antagonist | No efficacy |

PRESYNAPTIC GLUTAMATE RELEASE INHIBITORS

| | | |
|---|---|---|
| Lubeluzole | Sodium channel blockade, modulates | No efficacy |
| | nitric oxide synthase | |
| Fosphenytoin | Modulates sodium channel | No efficacy |
| Propentophylline | Inhibits adenosine transport | Trial stopped, adverse effects |

OTHER ORGANIC CHANNEL INHIBITORS

| | | |
|---|---|---|
| Clomethiazole | GABA agonist, modulates chloride | No overall efficacy, |
| | channel | improvement in large |
| | | infarcts, new trial ongoing |
| NBQX | AMPA-type glutamate antagonist | Trial stopped, adverse effects |
| Bay x3702 | Serotonin agonist | Ongoing phase III |
| GM-1 | Non-NMDA antagonist | No efficacy |
| Nalmefene | Kappa-selective opiate antagonist | No efficacy |
| BMS-204352 | Potassium channel agonist | No efficacy |

HEMODILUENT

| | | |
|---|---|---|
| DCL hemoglobin | Blood substitute | Phase II trial, adverse effects |

ANTOXIDANTS INHIBITING FREE RADICALS

| | | |
|---|---|---|
| Tirilazad | Inhibits lipid peroxidation | No efficacy |
| Ebselen | Glutathione peroxidase-like action | No efficacy |

DRUGS ACTING AGAINST LATE DAMAGE

| | | |
|---|---|---|
| Enlimomab | Anti-adhesion antibodies | No efficacy, adverse effect |
| Hu23F2G | Anti-adhesion antibodies | No efficacy |
| Piracetam | Membrane modulator | No overall efficacy, |
| | | new trial ongoing |
| Citicoline | Antioxidant, promotes | Possible benefit in |
| | phosphatidylcholine synthesis | medium-sized infarcts |
| bFGF | Neurotrophic factor | Trial stopped, adverse effects |

At present, there is no neuroprotectant drug that may be administered by the patient (even with the assistance from relatives) prior to hospital arrival. The reasons include: requirement of intravenous loading dose, adverse effects, narrow therapeutic time window, and potentially serious side effects in patients without stroke or with hemorrhagic stroke. Thus, treating a hemorrhagic stroke with clot fighting agents is likely to seriously exacerbate the damage.

### SUMMARY OF THE INVENTION

A method and system for timely treatment of a sudden onset of at least one neurological deficit in a subject is disclosed. The sudden onset of neurological symptoms is an indicator of a possible stroke, also termed a cerebrovascular accident. The method comprises administering an effective amount of melatonin to the subject immediately after the sudden onset of the at least one neurological deficit, and preferably the administration of melatonin is within three hours of the sudden onset of the at least one neurological deficit. Preferably, the effective amount of melatonin is at least about 200 mg and less than about 1000 mg, although for a small child or infant the effective amount of melatonin may be smaller than about 200 mg while for a large adult the effective amount of melatonin may be larger than about 1000 mg. It is expected that the effective amount of melatonin is no more than about 1500 mg in almost all cases.

The effective amount of melatonin may be delivered in multiple doses, preferably within about three hours of the sudden onset of neurological symptoms. The effective amount of melatonin may be delivered in combination with ongoing administration of aspirin to reduce the risk of blood clot formation, or administration of other agents to improve blood flow by reducing the formation of clots or dissolving blood clots. Some example agents affecting blood flow include estrogen, eNOS inducer, L-arginine, a statin, aspirin, tissue plasminogen activator, modified viper venom, and prourokinase. In addition, agents and devices for controlling and regulating blood flow may also be used in combination with melatonin to treat stroke or stroke-like events.

The method and system also include administering the effective amount of melatonin in response to detecting neurological changes with the assistance of at least one of computer assisted tomography scans, magnetic resonance imaging, and electroencephalogram recordings. Such monitoring may be advisable for subjects adjudged at high risk for stroke or stroke like events, and even become economically acceptable with technological improvements.

The effective amount of melatonin can be administered by many methods including one or more of oral delivery in liquid or solid form, enteral delivery via a feeding tube in liquid or powder form, intravenous injection or infusion, absorption through mucosal membrane such as rectal or buccal mucosa, and a transdermal patch.

### DETAILED DESCRIPTION OF THE INVENTION

The disclosed invention encompasses a method and system for timely treatment of a sudden onset of at least one neurological deficit in a subject is disclosed. The sudden onset of neurological symptoms is an indicator of a possible stroke, also termed a cerebrovascular accident. The inventors have discovered that the administration of melatonin, a naturally produced substance by the pineal gland, which is known to be safe from extensive use, shortly after a stroke serves to protect cerebral tissue from ischemia related damage. Since melatonin can be administered safely in a wide dose range (up to at least 50 mg/kg have been tested), it is possible to administer it even in cases of a suspected stroke.

Based on laboratory observations, the method comprises administering an effective amount of melatonin to a subject immediately after a sudden onset of at least one neurological deficit, and preferably the administration of melatonin is within three hours of the sudden onset of the neurological deficit. Preferably, the effective amount of melatonin is at least about 200 mg and less than about 1000 mg, although for a small child or infant the effective amount of melatonin may be smaller than about 200 mg while for a large adult the effective amount of melatonin may be larger than about 1000 mg. Typically, the amount of melatonin may be about 5 mg/kg to about 15 mg/kg, although melatonin at 50 mg/kg is effective as well. It is expected that the effective amount of melatonin can be no more than about 1500 mg per individual for almost all human subjects.

The effective amount of melatonin may be delivered in multiple doses, preferably within about three hours of the sudden onset of neurological symptoms. The delivered melatonin may be in combination with ongoing preventive administration of melatonin. The effective amount of melatonin may be delivered in combination with ongoing administration of aspirin to reduce the risk of blood clot formation, or administration of other agents to improve blood flow by reducing the formation of clots or dissolving blood clots. Some example agents affecting blood flow include estrogen, eNOS inducer, L-arginine, a statin, aspirin, tissue plasminogen activator, modified viper venom, and prourokinase. In addition, agents and devices for controlling and regulating blood flow may also be used in combination with melatonin to treat stroke or stroke-like events.

The laboratory observations in support of the disclosed invention include experiments with rats. In experimental studies, the control groups of animal received identical handling plus an intraperitoneal injection of the vehicle alone (i.e. without melatonin). There is additional experimental information on the beneficial mechanisms of melatonin and its ability to protect against in vitro ischemia.

For instance, pre-treatment with a single intraperitoneal (i.p.) injection of melatonin at doses between about 5 and about 15 mg/kg significantly reduced the infarct volume by about 40% at about 72 hours without affecting the systemic hemodynamic parameters and regional cerebral blood flow in both permanent and 3-hour endovascular middle cerebral artery occlusion (MCAO) stroke models in adult Sprague-Dawley rats. Indeed melatonin treatment was effective when the single injection of melatonin at about 5 mg/kg was commenced at 1 hour or less after onset of ischemia induced by a 3-hour endovascular MCAO in adult Sprague-Dawley rats. Addition of the second and third doses at 24 and 48 hours of ischemia tended to achieve increased reduction in infarct volume but failed to extend the treatment time window beyond about 3 hours of ischemia. Notably, there is no evidence of producing any harmful effects at doses up to about 50 mg/kg. In agreement with the observed safety of melatonin administration, large daily oral doses of melatonin at about 300 mg for about 4 months have been shown to inhibit ovulation in women without significant side effects.

It should be noted that although the invention has been described here in the context of the particular amounts of melatonin, the disclosed amounts are not intended to provide upper or lower limits for the effective amounts of melatonin. Variations that are apparent to one of ordinary skill in the art are also intended to be included in the scope of the invention. Moreover, although the mechanisms described herein represent the current view, it is not intended that the invention be bound by any particular theory for the mechanism of melatonin based protection, either alone or in combination with other substances such as anti-clot and anti-inflammatory factors or devices. Some additional details of interest are provided in the following references, all of which are hereby incorporated by reference in their entireties.

References

1. Acuna-Castroviejo D, Martin M, Macias M et al. Melatonin, mitochondria, and cellular bioenergetics. J Pineal Res 2001; 30:65-74.

2. de Butte M, Fortin T, Pappas B A. Pinealectomy: behavioral and neuropathological consequences in chronic cerebral hypoperfusion model. Neurobiol Aging 2002; 23:309-317.

3. Cheung R T. The utility of melatonin in reducing cerebral damage resulting from ischemia and reperfusion. J Pineal Res 2003; 34: 153-160.

4. Cho S, Joh T H, Baik H H et al. Melatonin administration protects CA1 hippocampal neurons after transient forebrain ischemia in rats. Brain Res 1997; 755:335-338.

5. Cuzzocrea S, Costantino G, Gitto E et al. Protective effects of melatonin in ischemic brain injury. J Pineal Res 2000; 29:217-227.

6. Cuzzocrea S, Reiter R J. Pharmacological action of melatonin in shock, inflammation and ischemia/reperfusion injury. Eur J Pharmacol 2001; 426:1-10.

7. Furlan A, Higashida R, Wechsler L et al. Intra-arterial prourokinase for acute ischemic stroke. The PROACT II study: a randomized controlled trial. Prolyse in Acute Cerebral Thromboembolism. JAMA 1999; 282:2003-2011.

8. Guerrero J M, Reiter R J, Ortiz G G et al. Melatonin prevents increases in neural nitric oxide and cyclic GMP production after transient brain ischemia and reperfusion in the Mongolian gerbil (Meriones Unguiculatus). J Pineal Res 1997; 23:24-31.

9. Joo J Y, Uz T, Manev H. Opposite effects of pinealectomy and melatonin administration on brain damage following cerebral focal ischemia in rat. Restor Neurol Neurosci 1998; 13:185-91.

10. Kilic E, Ozdemir Y G, Bolay H et al. Pinealectomy aggravates and melatonin administration attenuates brain damage in focal ischemia. J Cereb Blood Flow Metab 1999; 19:511-516.

11. Laufs U, Endres M, Stagliano N et al. Neuroprotection mediated by changes in the endothelial actin cytoskeleton. J Clin Invest 2000; 106:15-24.

12. Letechipia-Vallejo G, Gonzalez-Burgos I, Cervantes M. Neuroprotective effect of melatonin on brain damage induced by acute global cerebral ischemia in cats. Arch Med Res 2001; 32:186-192.

13. Liao, J K. Statins: Are there benefits beyond Cholesterol lowering? Manuscript (last modification on August, 2002), obtained from website http://www.fcmsdocs.org/Conference/11th/Statins%20%20Are%20There%20Benefits%20Be yond%20Cholesterol%20Lowering.pdf, attached herewith.

14. Manev H. Uz T, Kharlamov A et al. Increased brain damage after stroke or excitotoxic seizures in melatonin-deficient rats. FASEB J 1996; 10:1546-1551.

15. Martinez-Vila E, Irimia-Sieira P. Current status and perspectives of neuroprotection in ischemic stroke treatment. Cerebrovase Dis 2001; 11 [suppl 1]:60-70.

16. Mesenge C, Margaill I, Verrecchia C et al. Protective effect of melatonin in a model of traumatic brain injury in mice. J Pineal Res 1998; 25:41-46.

17. Pei Z, Pang S F, Cheung R T. Pretreatment with melatonin reduces volume of cerebral infarction in a rat middle cerebral artery occlusion stroke model. J Pineal Res 2002; 32:168-172.

18. Pei Z, Pang S F, Cheung R T. Administration of melatonin after onset of ischemia reduces the volume of cerebral infarction in a rat middle cerebral artery occlusion stroke model. Stroke 2003; 34:770-775.

19. Reiter R, Tang L, Garcia J J et al. Pharmacological actions on melatonin in oxygen radical pathophysiology. Life Sci 1997; 60:2255-2271.

20. Reiter R J, Tan D X, Manchester L C et al. Biochemical reactivity of melatonin with reactive oxygen and nitrogen species: a review of the evidence. Cell Biochem Biophys 2001; 34:237-256.

21. Sherman D C, Atkinson R P, Chippendale T et al. Intravenous ancrod for treatment of acute ischemic stroke: the STAT study: a randomized controlled trial. Stroke Treatment with Ancrod Trial. JAMA 2000; 283:2395-2403.

22. Sinha K, Degaonkar M N, Jagannathan N R et al. Effect of melatonin on ischemia reperfusion injury induced by middle cerebral artery occlusion in rats. Eur J Pharmacol 2001; 428:185-192.

23. The National Institute of Neurological Disorders and Stroke rt-PA Stroke Study Group. Tissue plasminogen activator for acute ischemic stroke. N Eng J Med 1995; 333:1581-1587.

24. Voordouw B C, Euser R, Verdonk R E et al. Melatonin and melatonin-progestin combinations alter pituitary-ovarian function in women and can inhibit ovulation. J Clin EndocrinolMetab 1992; 74:108-117.

We claim:

1. A method for treating a sudden onset of at least one neurological deficit in a subject comprising:
administering an effective amount of melatonin to the subject immediately after the sudden onset of the neurological deficit.

2. The method of claim 1, wherein the effective amount of melatonin is at least about 200 mg.

3. The method of claim 1, wherein the effective amount of melatonin is no more than about 1500 mg.

4. The method of claim 1 further comprising the step of delivering the effective amount of melatonin in multiple doses.

5. The method of claim 1, wherein the effective amount of melatonin is delivered within about three hours of the sudden onset of neurological symptoms.

6. The method of claim 1 further comprising administering the effective amount of melatonin in response to detecting neurological changes with the assistance of at least one of computer assisted tomography scans, magnetic resonance imaging, and electroencephalogram recordings.

7. The method of claim 1 further comprising administering the effective amount of melatonin in combination with preventive administration of melatonin.

8. The method of claim 1 further comprising administering the effective amount of melatonin in combination with at least one agent for improving blood flow.

9. The method of claim 8, wherein the at least one agent for improving blood flow is selected from estrogen, eNOS inducer, L-arginine, a statin, aspirin, tissue plasminogen activator, modified viper venom, and prourokinase.

10. The method of claim 8, wherein the agent for improving blood flow is administered separately from the administration of the effective amount of melatonin.

11. The method of claim 1, wherein the effective amount of melatonin is administered by one or more methods selected from oral delivery in liquid or solid form, enteral delivery via a feeding tube in liquid or powder form, intravenous injection or infusion, absorption through mucosal membrane such as rectal or buccal mucosa, and a transdermal patch.
